(19) United States
(12) Plant Patent Application Publication
(10) Pub. No.: US 2013/0276181 P1
(43) Pub. Date: Oct. 17, 2013

(54) Grapevine 'Sheegene-17'

(50) ;

(71) Applicant: Timothy P. Sheehan, Vancouver, WA (US)

(72) Inventor: Timothy P. Sheehan, Vancouver, WA (US)

(73) Assignee: Sheehan Genetics, LLC

(21) Appl. No.: 13/506,354

(22) Filed: Apr. 12, 2012

(51) Int. Cl. A01H 5/00

(52) U.S. Cl. PLT/207

(57) ABSTRACT

A new and distinct variety of grapevine characterized by the production of medium, green-colored seedless grapes that mature in late August when grown in the San Joaquin Valley of Central California. The grapes of this new variety are produced on strong woody stems and branches and are well adapted to commercial handling.

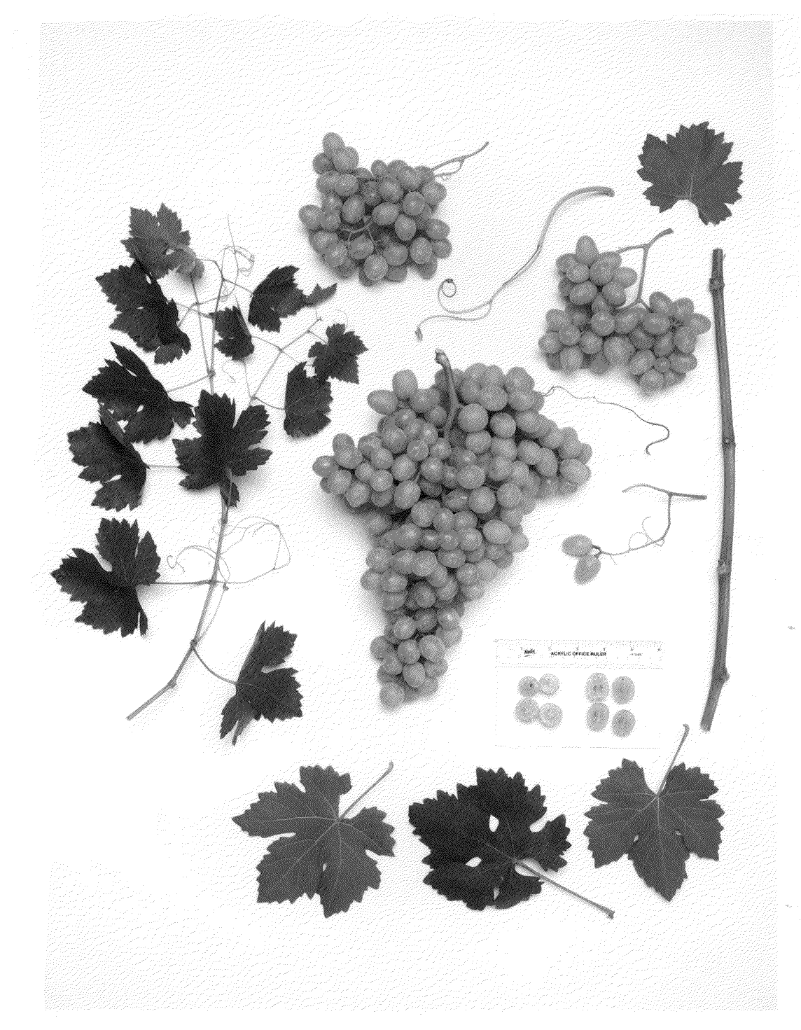

### CROSS REFERENCE TO RELATED APPLICATIONS

The claimed plant is not subject of a related application.

### STATEMENT OF ANY FEDERALLY-SPONSORED RESEARCH AND DEVELOPMENT

The claimed plant is not subject of Federally-sponsored research or development.

### LATIN NAME OF THE GENIUS AND SPECIES OF THE PLANT CLAIMED

The claimed plant relates to a new and distinct variety of Vitis vinifera to be known as ‘Sheegene-17’.

### VARIETY DENOMINATION

The new variety of Vitis vinifera is the result of hybridization of ‘Princess’ (unpatented), the pollen parent, and ‘Red Globe’ (U.S. Plant Pat. No. 4,787), the seed parent. The new variety was asexually propagated by Timothy P. Sheehan in 2000 Spring? in a Vitis vinifera variety block located East of Fowler, California. The new variety was planted as rooted cuttings in the dormant season of 2006 in a field located several miles South of McFarland, Calif. The new variety produces a medium, green-colored seedless grape with a sweet flavor. The new variety has been shown to maintain its distinguishing characteristics through asexual propagation.

### BACKGROUND OF THE INVENTION

The present invention relates to a new and distinct variety of grapevine that produces a medium, green-colored seedless grape that matures in late August when grown in the San Joaquin Valley of Central California. The new invention somewhat resembles ‘Thompson Seedless’ (unpatented).

### SUMMARY OF THE INVENTION

The ‘Sheegene-17’ grapevine is characterized by producing a medium, green-colored seedless grape that has a sweet flavor and it is productive and mature for harvesting and shipment in late August when grown in the San Joaquin Valley of Central California. The new variety can be compared to ‘Thompson Seedless’ (unpatented) but the grapes produced by the new variety are larger and mature two to three weeks later, among other distinguishing characteristics.

### BRIEF DESCRIPTION OF THE DRAWINGS

The accompanying drawing is a color photograph of the new variety of grapevine displaying leaves, tendrils, shoots, mature grapes, late berry cluster, mature berry cluster, and four grapes cut in half. Across the top portion of the photograph is a late berry cluster with a young shoot to the right of the cluster. Directly below the cluster and to the left is an immature shoot with leaves. At the center of the photograph is a mature berry cluster with a mature cane and nodes to the right of the cluster. Just below the mature berry cluster are four grapes cut longitudinally and horizontally displaying the heart and pulp. Below the cut grapes are leaves displaying the upper and lower surfaces.

### DETAILED BOTANICAL DESCRIPTION

Referring more to the horticultural description of the new and distinct variety of grapevine, the following has been observed under the ecological conditions prevailing at the origin vineyard located near McFarland, Calif., in the San Joaquin Valley of Central California. All major color descriptions are by reference to the Dictionary of Color by Maerz & Paul, First Edition, published in 1930. Common colors names are also used in several instances.
Vine:
   Size.—Medium for a seven year-old vine.
   Vigor.—Very good.
   Chilling requirements.—Normal for grapevines in the lower San Joaquin Valley of Central California.
   Productive capacity.—Very good.
   Regularity of bearing.—Regular.
Trunk:
   Size.—Medium; 8 inches [20.32 cm] in circumference, 15 inches [38.10 cm] above ground.
   Surface texture.—Rough and shaggy.
   Color of bark.—Pl.3 G9 Kaffa Hampshire Brown.
   Mature cane color.—Pl.7 L8 Mauve Taupe Copra.
   Nodes.—Four on cane, 15 inches [38.10 cm] long.
   Length between nodes.—4 inches [10.16 cm].
   Shoot length.—6 to 6.5 inches [15.24 to 16.51 cm].
   Shoot shape.—Droopy.
   Tendrils number/location.—Single at node.
   Tendrils length.—1.5 to 3.75 inches [3.81 to 9.53 cm].
   Tendrils locale.—At nodes.
   Tendrils form.—Bifurcate.
   Tendrils texture.—Firm.
   Bud shape.—Convex.
   Bud size.—0.24 to 0.28 inches [6 to 7 mm] wide; 0.24 to 0.28 inches [6 to 7 mm] long.
   Bud fruitfulness.—Very good.
Branches:
   Size.—Medium.
   Cordons.—26.5 inches [67.31 cm] on one side of trunk ; 30.0 inches [76.20 cm] on the other side of trunk.
   Surface texture.—Slightly rough.
   Color (one year or older branches).—Pl.6 K9 Havana Rose.
   Color (immature branches).—Pl.19 K6 Piquant gr.
Leaves:
   Size.—Medium.
   Density.—Dense.
   Average length.—3.75 to 5.25 inches [9.53 to 13.34 cm].
   Average width.—5.00 to 6.5 inches [12.70 to 16.51 cm].
   Form.—Pentagonal.
   Texture (upper surface).—Smooth.
   Texture (lower surface).—Slightly rough.
   Color (upwardly disposed surface).—Pl.23 J8 Mt. Vernon gr.
   Color (downwardly disposed surface).—Pl.21 K8 Teak fern.
   Color (leaf Vein).—Pl.17 L3 Endive.
   Marginal form.—Toothed.
   Leaf margin.—Slightly undulate.
   Leaf vein thickness.—0.08 inches [2 mm].
   Stem glands.—None.
   Petiole size.—Moderate.
   Petiole length.—2.75 to 5.25 inches [6.99 to 13.34 cm].
   Petiole thickness.—0.12 inches [3 mm].
   Petiole color.—Pl.6 L8 Dark Cardinal Rose Lustre.
   Stem glands.—None.
   Stipules.—None.
   Lobes (average).—Four.
   Stipules.—None.
   Tooth size.—0.31 to 0.39 inches [8 to 10 mm] in width; 0.31 inches [8 mm] in length.
   Tooth number.—3 to 4 between lobes.
   Tooth shape.—Convex.
Inflorescence:
   Size.—8.00 to 9.00 inches [20.32 to 22.86 cm].
   Number borne per spur.—Two.
   Number borne per vine.—Thirty.
Flowers:
   Flower buds (size).—Small.
   Flower buds (surface).—Glabrous.
   Flower buds (quantity).—Moderate.
   Date of bloom.—Apr. 15, 2007.
   Date of full bloom.—Apr. 21, 2007.
   Size of bloom.—0.20 to 0.24 inches [5 to 6 mm].
   Petal (calyptra) color.—Pl.19 D2 Reed.
   Berry color.—Pl.18 E6 Oriental Tiber gr.
   Petals size.—0.08 to 0.12 inches [2 to 3 mm].
   Stamen (anther) color.—Bl White.
   Date of first visible berries.—May 1, 2007.
Fruit:
   Solids.—16.9 Brix.
   Acids.—0.61.
   Sugar/acid ratio.—27.7.
   Juice pH.—3.51.
   Seeds.—None.
   Capstem pedicel.—0.24 to 0.39 inches [6 to 10 mm].
   Berry weight.—5.68 grams (0.20 oz).
   Cluster size.—Medium.
   Cluster average length (not including stem).—10 inches [25.40 cm].
   Cluster average diameter.—6.25 inches [15.88 cm].
   Cluster average weight.—965.6 grams (34.06 oz).
   Compactness.—Compact.
   Cluster form.—Conical.
   Stem (length).—1.5 inches (3.81 cm).
   Stem caliper.—0.28 inches [7 mm].
   Berry size.—Medium.
   Berry form.—Ovate.
   Berry numbers (average).—Approximately 166 berries per bunch.
   Berry size (average dimension along longitudinal axis).—0.91 to 1.14 inches [26 to 29 mm].
   Berry Size (average dimension along transverse axis).—0.83 to 0.91 inches [21 to 23 mm].
Skin:
   Skin thickness.—Medium.
   Texture.—Semi-tough.
   Tendency to crack.—Slight.
   Ground color.—Pl.19 C6 Pistachio gr. Pistache.
   Pulp color.—Pl.19 C6 Pistachio gr.
   Lenticels.—None.
Flesh:
   Flesh color.—From Pl.19 C16 Pistachio gr. Pistache.
   Juice production.—Moderate.
   Flavor.—Very good.
   Aroma.—Very mild.
   Texture.—Firm.
   Ripening.—Even.
   Eating quality.—Very good.
   Use.—Fresh market.
   Keeping quality.—Good.
   Resistance to disease.—Unknown.
   Harvesting.—Late August in the San Joaquin Valley of Central California.
   Shipping and handling qualities.—Very good.

Having thus described and illustrated our new variety of grapevine, we claim:

1. A new variety of grapevine to be known as ‘Sheegene-17’ and parts thereof, substantially as illustrated and described, characterized principally by its production of medium, green-colored seedless grapes that mature two to three weeks later than ‘Thompson Seedless’ (unpatented) which it somewhat resembles.
